(19) United States
(12) Patent Application Publication
(10) Pub. No.: US 2024/0252757 A1
(43) Pub. Date: Aug. 1, 2024

(54) ADMINISTRATION DEVICE WITH IMPROVED USER GUIDANCE

(71) Applicant: Ypsomed AG, Burgdorf (CH)

(72) Inventors: Martin Bruegger, Bolligen (CH); Patrick Hostettler, Hasle (CH)

(21) Appl. No.: 18/619,353

(22) Filed: Mar. 28, 2024

Related U.S. Application Data
(63) Continuation of application No. PCT/EP2022/077118, filed on Sep. 29, 2022.

(30) Foreign Application Priority Data
Oct. 5, 2021 (EP) 21200856.9

(51) Int. Cl. A61M 5/31

(52) U.S. Cl. CPC A61M 5/3146; A61M 2205/50

(57) ABSTRACT

An administration device and a method for a guided operation of an administration device for a medication includes: a pre-selection means by means of which a pre-selected quantity can be mechanically or electronically set and/or stored, and a controller containing a timer configured such that, when an operating means is actuated, the timer continuously measures a duration between when the output means is no longer moved by the drive to when the operating means is transferred to the non-actuated state. The controller identifies the discharge quantity as a priming quantity if the duration is shorter than a defined threshold duration.

### CROSS-REFERENCE TO RELATED APPLICATIONS

This application is a continuation of International Application No. PCT/EP2022/077118, filed on Sep. 29, 2022, which in turn claims priority to European Patent Application No. 21200856.9, filed on Oct. 5, 2021, each of which is incorporated by reference herein, in the entirety and for all purposes.

### TECHNICAL FIELD

Administration devices, administration systems and methods are provided for the generation, evaluation, storage, and communication of data in connection with dosing and administration of medications as part of a treatment, which may be over an extended or indeterminate period of time.

### BACKGROUND

Discharges with administration devices and/or administration systems are referred to as a treatment-related discharge administration, administration for short, if they take place in the body, for example, the subcutaneous tissue of a patient, during treatment. In order to ensure accurate dosing, the administration of air in place of medication must be precluded and, in order to check the reliable function of the device, it is regularly also necessary to carry out discharges in which the needle remains outside the patient's body, or is not inserted. Such discharges, in which the user can observe the event at the needle tip, are referred to as a priming discharge, priming or also “air shot” for short. The medication thus discharged is dispensed (as a priming quantity) accordingly to the surroundings or directed into a vessel provided for this purpose and is not treatment-related. In contrast, in the case of treatment-related administration, the needle having a hollow cannula is inserted, for example, into the subcutaneous tissue of a patient before the discharge. The needle should only be removed from the injection site after the discharge (of an administration quantity) and after a specified dwell time. This specified dwell time allows sufficient diffusion or distribution of the medication in the intended dose in the tissue of the patient. The treatment plan or the device instructions or a patient information leaflet of the medication can specify a prescribed duration or minimum duration for the dwell time, for example 10 seconds after the end of an injection. In this case, it can be difficult for a user or a patient to determine the end of the discharge and consequently to correctly measure the start and also the duration of the specified dwell time.

Users or patients who use multi-use injection devices, such as insulin pens, usually follow an administration or treatment plan. In the course of the day, such a plan can provide a number of administrations, each with different doses at certain points in time and/or opportunities. Users or patients are advised to keep precise records of all administrations, such as the treatment-related administration discharges. This is laborious and susceptible to human error.

Publications are known from the prior art which disclose devices or methods for priming administration devices or keeping priming and administration separate.

Thus, WO 15066522 A1 discloses an exemplary embodiment that can prepare a user before an injection. The user begins to vent the injection device in that the user orients the injection device and thus the outlet opening of the liquid container upwards. The rotation of the injection device into another orientation can be detected by a sensor, such as an accelerometer or another rotation sensor within the injection device. This rotation of the liquid outlet opening upwards can enable air to rise toward the upper side of the device and a possibly present needle. Therefore, the detection of the rotation of the liquid outlet opening into the predetermined orientation, in which the liquid outlet opening points upwards, is contained in the aspects of the teaching shown. Once the liquid outlet opening is rotated upwards, the user can tap a medication reservoir to cause remaining air bubbles to rise. In one exemplary embodiment, a mobile device can display an instruction to a user to tap a medication reservoir. As soon as it has been detected that the injection device is oriented upwards with the outlet opening of the liquid container, the injection system can activate an input, such as a priming button or a trigger. The user then carries out a venting function by using, for example, a touchscreen on a mobile device connected to the injection device by selecting or activating the vent button or the trigger. The venting function then causes the injection device to be vented, wherein the air is pressed out of the outlet opening of the liquid container by the liquid.

WO 1117404 A2 discloses a display, which can be used to supply information about the priming of medication administration or possibly to display the orientation and/or relative position of the device. For example, a microelectromechanical accelerometer is provided in the device, so that the device has the intelligence to detect whether the user is using the device to perform a safety or priming discharge (i.e., the distal end of the device points upwards) or whether the user is using the device to perform a dose administration step (i.e., the distal end of the device points downwards). The display can also be used as a log or lifestyle calendar and perhaps communicate with the glucose monitor of the patient and may store and display glucose data. A dwell time could also be displayed on the display after a dose is dispensed, which may be proportional to the dose size. The display could display whether the device is activated, i.e., ready to deliver a dose, and there could also be an indication if the dose is outside the expected thresholds.

In one embodiment of the publication WO 12160157 A1, the control device can be operated such that it defines the priming volume or the profile of the intermittent dose on a random basis. This can be done using a “random seed,” i.e., a random number that is generated by a random number generator and is used when calculating the size or volume of one or more intermittent doses. The priming volume can be selected from a dosing menu, optionally depending on the dwell time (i.e., how long the dosing key is pressed) of the user activation of the user interface. A basic pre-configuration can be set in the priming menu. These options may be provided if needles of different sizes are considered for use with the device. Furthermore, a method for controlling a handheld insulin injection pen for discharging one or more drugs is shown, wherein the pen comprises at least one priming mode and a drug dispensing mode for administering the one or more drugs, wherein the method comprises: controlling the pen to perform an initialization process; and administering a plurality of intermittent doses of a medication depending on the activation of the priming process by a user interface, wherein the plurality of intermittent doses comprises a predetermined sequence of priming doses.

The disclosure in the publication WO 2021/041378 A1 relates to methods for determining dose information of injections. In this case, data of an injection event group related to one or more injection events on one or more devices are accessed. The injection event group is processed to determine an actually administered dose of a particular injection event based on one or more of the following criteria: how the injection event correlates with a device change; how the injection event can be classified with respect to the dose size with other events in the group; which position it assumes in the injection event group and/or with which duration it is spaced apart from adjacent injections of the injection event group.

WO 2021/116052 A1 discloses an administration device for medications having a dose dispensing button and an “end of dose” or “0U” switch or sensor, which is closed or activated shortly before a running discharge is ended, i.e., before a stop mechanically terminates the running discharge. In this case, there is always the possibility that the user releases the dose dispensing button too early, and thus ends a discharge too early. A delay time is therefore provided which mitigates this problem. This is done by aligning the delay time to the human reaction time (or slightly below). Thus, it is largely ensured that the doses are recorded as fully delivered before a user can react to an “end of dose” signal and release the dose dispensing button. In other cases where the user releases the dose dispensing button when the discharge is running, the dose is recorded as incompletely discharged.

The publication US 2019/0192778 A1 discloses a data acquisition device which can be attached to a dose selector of an administration device. An optical switch can determine the start and end time of an administration process. A timer is contained which can measure the time which has elapsed between two injections.

The publication WO 2019/121617 A1 discloses an administration device comprising a computer and a contactless sensor which can detect positions or actuations of movable parts in the administration device, whereby the user can be informed with respect to the beginning and end of a predetermined holding time by means of an indication output on a display

The publication WO 2021/095003 A1 discloses a sensor module for a medication administration device, which module can be attached to said device. The sensor module comprises a sensor which can detect at least one of various physical variables. A computer and a communication interface allow the detection and wireless transmission of the data to an external location. A sensor and a timer/clock can determine the point in time of an injection. The noises during use of the administration device can be detected with an acoustic sensor and evaluated by the computer, whereby it can be assessed whether the rules of the treatment are being followed and the medication is being correctly administered.

For reliable, safe dosing or treatment, it is important to know the effectively administered quantity of a medication. Discharges which are used merely for venting must not be counted and registered as an administered quantity. The solutions shown in the prior art cited above with, for example, position sensors or gyroscopes are unreliable, since the device can be tilted or pivoted from the prescribed position in space for a venting process. Likewise, it cannot be ruled out that an effective quantity is administered in such a position. Allowing venting only in an operating mode specifically provided for this purpose is complicated and carries the risk of incorrect application. Detecting a venting using a small quantity limits the possibilities of efficient venting and a wide administration range. Methods that are based on certain predefined patterns can only be applied in predetermined cases and are susceptible to human inaccuracies.

Such parameters of methods and devices that are known or can be determined from the prior art relate to certain predefined conditions or predefined criteria, i.e., to ideal or foreseeable administration environments and/or administration scenarios or to certain predefined starting values, and are therefore often not suitable or are insufficiently suitable or cannot be applied reliably to an instantaneous, i.e., immediately resulting administration scenario or to an immediately resulting user need or treatment requirement.

### SUMMARY

An underlying object of the present invention is to provide an administration device for parenteral administration of a medication and methods which simplify the reliable use of the device and thereby increase safety during application. It is further an object of the present invention to provide devices and methods which can be applied reliably to an immediately resulting administration scenario or to an instantaneous user need or treatment requirement.

The object is achieved by devices and methods according to the independent claims. Developments are evident from the illustration of the embodiments and/or from the disclosure. The developments and aspects shown can be combined largely independently of one another or as desired.

In one aspect of the present invention, an improved distinction of priming and administration is shown. Further aspects of the invention relate to their application within the scope of technical systems and methods which are used within the scope of medication treatments, for example in diabetological or diabetes management systems which, in addition to the administration devices, can further have glucose monitors, e.g. for continuous glucose measurement, remote controls, bolus calculators, and host systems. The aspects of the present invention support the user in the error-free and simple handling of such systems, which leads to improved treatment results.

Furthermore, the present invention can improve the accuracy of a calculation of the “insulin on board” IOB by optimizing the quality of the input variables for the IOB calculation. The calculated IOB value makes a statement about the active insulin in the body of the patient at a specific point in time, where the accuracy of the calculation also benefits from the quality of the improved recording of the administration history according to the invention, in that the present invention improves and automates the registration of the points in time and the administered quantities. Finally, a precise IOB value leads to a more precise bolus suggestion and thus to an improved treatment.

For example, the present invention can improve the “time in range” TIR, i.e., the duration within the glycemic target range. With the increasing prevalence of diabetes technology, the TIR as an alternative and/or supplement to HbA1c is leading to a paradigm shift in diabetology. Continuous glucose measurement has made it possible to identify patterns—in relation to rising/falling values at certain times of day or after certain meals. These can be used to adapt the treatment. The “time in range” (TIR, time within the target range) is an indicator of the duration of the normo-, hypo- and hyperglycemic phases. In analogy to the assessment of the blood pressure setting, this parameter can be expressed by 2 values:
the percentage of time within the target range 70-180 mg/dl (3.9-10.0 mmol/L) and
the time in the hypoglycemic range<70 mg/dl/<3.9 mmol/L
   Examples of TIR parameters: 52/15% or 84/0%

Furthermore, in one of its aspects, the present invention can simplify the operation of an administration device through intuitive user guidance in that correct dose settings and correct administration times can be qualitatively signaled directly on the administration device. A simple, clear operating aid is thereby offered before and during the administration, and a quantitatively (numerical) setting of dose values is optional or completely omitted.

The present invention relates to an administration device for parenteral administration of a medication, having a pre-selection means, by means of which a pre-selected quantity can be set and/or stored mechanically or electronically, a manually operable drive or a releasable motor drive that moves an output means (piston rod, threaded rod, segment rod, flange, bellows, piston, roller, etc.) relative to a container (carpule, syringe, bag, etc.), which can be inserted into the administration device and which contains a medication, until a discharge quantity, which corresponds to the movement of the output means and which is discharged out of the container through a needle and/or hollow cannula, reaches the pre-selected quantity set or stored by the pre-selection means or another stop criterion occurs, a manually actuatable operating means (pushbutton, slide, button, switch, etc.), which can have a non-actuated and an actuated state and which can operate and/or release the drive in the actuated state, an electronic controller (e.g., a control unit) containing a time measuring device (e.g., timer), where the time measuring device is configured such that when the operating means is actuated, the time measuring device continuously measures the duration from the point in time at which the output means is no longer moved by the drive to the point in time at which the operating means is transferred to the non-actuated state, such as to continuously measure at the longest until the point in time at which the operating means is transferred to the non-actuated state.

The duration continuously determined and finally reached in this way forms the starting point for developments of the present invention. The duration ultimately reached can also be referred to as a holding duration and can be determined substantially by the user or the patient by actuating the operating means, as a result of which a running and a subsequent evaluation, characterization or the intended purpose of a discharge can be influenced or is influenced deliberately or intuitively.

Another stop criterion can, for example, be or supply a mechanical zero stop, a limit switch, an empty container or a non-actuated operating means (i.e., when the operating means changes to the non-actuated state when the drive is running) or a device error, for example, a drive error or a sensor error or a detected blockage or blocking of the needle or the discharge path.

The administration device is further developed by the electronic controller being configured to characterize or identify the discharge quantity as a priming quantity as long as or if the duration is less than a predefined threshold duration and/or to characterize the discharge quantity as an administration quantity as soon as or if the duration is equal to or greater than the predefined threshold duration. The electronic controller can currently determine the relation of the duration to the predefined threshold duration at any time in order to determine whether a discharge is to be characterized or identified as priming or as administration. This has the advantage that a user or a patient intuitively indicates whether a single discharge was made for priming or as an administration through the provided handling of the administration device.

The predefined threshold duration is defined in the electronic controller as a value from a range from 1 to 20 seconds, or in a range from 2 to 5 seconds, or as 3 seconds, or can be specified variably from a controller (remote control, smartphone app, blood glucose monitor (BGM), continuous glucose monitor (CGM), PC, network, cloud, host, etc.). For example, the predefined threshold duration can be smaller than or correspond to a dwell time specified on the patient information leaflet for the medication, where a user is supported or guided by the administration device during the measuring of the dwell time and has the advantage that individual patient needs or special medical, pharmaceutical or physiological conditions relating to a variation of the dwell time can be taken into account without the reliability of the distinction between priming and administration being reduced.

A specified dwell time is defined as a value from a range of 0 to 20 seconds, or from a range of 5 to 12 seconds, or as 10 seconds, or can be specified variably from a controller. According to the invention, the specified dwell time can also be shorter or longer than a dwell time defined according to the intended use. This has the advantage that individual patient needs or special medical, pharmaceutical or physiological conditions relating to a variation of the dwell time can be taken into account without the reliability of the distinction between priming and administration being reduced.

The electronic controller can determine the standstill of the output means due to one or more drive parameters (for example, current, counter-electromotive force (EMF), derivatives thereof) or by means of a limit switch or sensor or a rotary or linear encoder.

The electronic controller has a sensor, for example, an encoder or, with which the quantity of medication corresponding to the movement of the output means or the discharge quantity can be determined quantitatively.

The container is a cartridge with a stopper and septum. For example, such carpules can have a useful volume of 3.0 ml or 1.6 ml.

The electronic controller has a real-time clock, where events or status changes, such as discharge quantities, can be supplemented with a time and/or date stamp.

The real-time clock can be synchronized with a clock on the controller.

The electronic controller has a temperature sensor, where the temperature profile during operation and storage is monitored, and thus events or status changes can be supplemented with a temperature indication. This also allows the quality of the medication to be ensured by determining the temperature minima and maxima to which an inserted container or the medication contained therein is or was exposed. The temperature sensor is attached near and/or thermally coupled to the container.

The electronic controller has a memory which records at least the last discharge quantity corresponding to the movement of the output means as an administration quantity, provided that it is not characterized as a priming quantity. Where the discharge quantity or the administration quantity and/or the priming quantity can each be provided with a time stamp and/or date stamp and/or at least one further characterization or identification (medication, concentration, lot number, carpule number, temperature, geographical location, spatial orientation, conformity with the intended use, etc.).

The electronic controller has further memories for the sum of a plurality of administration quantities and/or priming quantities.

The electronic controller has further memories, each for a plurality of successive administration quantities and/or priming quantities, wherein the plurality of successive administration quantities and/or priming quantities can be provided with a time stamp and/or a characterization (medication, concentration, lot number, carpule number, temperature, geographical location, spatial orientation, etc.). This makes improved recording of the administration history possible.

The electronic controller can have a transceiver in order to establish a wireless connection to a controller, realized, for example, by Bluetooth Low Energy (BLE), near-field communication (NFC), ultra-wideband (UWB) technology, where this can be secured by suitable authentication and encryption.

The controller can be one of, for example, a remote control, smartphone app, BGM, CGM, PC, network, cloud, host, etc., or the controller can be a system of a plurality of remote control, smartphone app, BGM, CGM, PC, network, cloud, host, etc. The controller can also be merely a CGM device in normal operation, where the interaction, for example, with a smartphone app can be completely dispensed with, or this is necessary only when a new CGM device or glucose sensor is put into operation. This allows the user or the patient to use a simpler application even in situations where, for example, a smartphone cannot be available. Alternatively, the functionality of the controller can also be implemented in the electronic controller in the administration device itself. This allows, for example, the communication/interaction with a continuous blood glucose monitor CGM without further system components. In addition, the administration device according to the invention can thereby also be operated temporarily or continuously autonomously.

The controller can receive a default value for a next discharge from the controller via the secure wireless connection; it can additionally be indicated whether the default value relates to an administration or a priming. Such a default value can be calculated, for example, by a bolus calculator implemented in the controller on the basis of inputs and the administration history as well as individual parameters. For example, one value for the currently measured glucose level and one for the energy content of the immediately imminent meal can be entered on a smartphone app. From these inputs, the IOB, and further individual parameters, the bolus calculator calculates an insulin dose which can be sent by the user or the patient by an input confirmed as a default value to the administration device. Such a bolus calculator or the controller is also able to spontaneously propose correction boluses due to, for example, the current CGM data and/or to transmit it to the administration device.

The electronic controller can transmit the most current stored administration quantity and/or priming quantity to the controller through the secured wireless connection and/or can transmit the plurality of successive administration quantities and/or priming quantities including associated time stamps and characterizations.

The administration device has a scale (printed sleeve with pointer, LCD, LED, e-paper, etc.) which can quantitatively display the quantity currently pre-selected by the pre-selection means, e.g. as a number of units.

Configured in that, in addition or alternatively, the quantity currently pre-selected by the pre-selection means can be displayed by the controller, for example, on a screen of a smartphone, which improves the readability and operating safety.

Advantageously developed in that the administration device and/or the controller has a display means (multicolored or monochrome LED, LCD, e-paper, voice output, buzzer, etc.) which can signal, controlled by the electronic controller, that the current pre-selected quantity set by the pre-selection means is at least one of smaller than, equal to, somewhat greater than, much greater than the default value and/or the discharge quantity has reached the pre-selected quantity pre-selected or stored by the pre-selection means and/or the current duration is at least one of smaller than, equal to, somewhat greater than, much greater than the predefined threshold duration and/or a specified dwell time, and/or a stop criterion has occurred, for example, an error. As a result, the correctness of the pre-selected quantity and/or the characterization or the treatment relevance of the discharge and/or the dwell time of the needle in the tissue according to the intended use can be displayed qualitatively, which facilitates user guidance and increases the reliability of the administration device.

An administration can be characterized or identified as compliant in the event of a dwell time of the needle in the tissue according to the intended use or if the duration is greater than or equal to the specified dwell time. The measured duration can be used currently or retroactively to determine whether an administration is compliant with the intended use.

Configured in that, in the event of a duration of less than the specified dwell time, the administration quantity is reduced by the quantity of medication that is therefore not diffused into the tissue or the administration or administration quantity can be nullified.

The display means has, for example, one or more multicolored LEDs, which can be controlled to modulate emitted light color and/or light intensity and/or pulse duration or duty factor. Alternatively or additionally, the display means can also have one or more single-color LEDs. Such a display means can, for example, have one, or a plurality of, the following signals:

Operating means (button) is not actuated:

| Color/duty factor | White | Green | Red |
|---|---|---|---|
| Continuous | Pre-selected | Pre-selected | Pre-selected |
| | quantity < > | quantity = Default | quantity >> |
| | Default value | value | Default value |
| Flashing | | | Device error |

Operating means (button) actuated:

| Color/duty factor | White | Green | Red |
|---|---|---|---|
| Continuous | | Duration >= | |
| | | Threshold | |
| | | duration/dwell time | |
| | | (administration | |
| | | terminated) | |
| Flashing | Pre-selected | Pre-selected | Pre-selected |
| | quantity < > | quantity = Default | quantity >> |
| | Default value, | value, Duration < | Default value, |
| | Duration < | Threshold | Duration < |
| | Threshold | duration/dwell time | Threshold |
| | duration/dwell | | duration/dwell |
| | time | | time |

The display means signals, with a distinguishable duty cycle or another modulation, whether the predefined threshold duration is not reached or is exceeded. In this way, the user can recognize whether a current discharge is characterized or identified as priming or as administration.

The display means signals, with a distinguishable duty cycle or another modulation, whether the specified dwell time is not reached or is exceeded. In this way, the user can recognize whether a current discharge has been administered in a compliant manner, for example, according to the intended use.

The administration device and/or the controller has a second display means, with which the state of the secure wireless connection can be signaled, for example, a blue LED.

The pre-selected quantity settable or set by the pre-selection means or the stored default value can be changed, defined or limited from the controller.

The default value can be taken from a plan or schedule stored in the administration device in a time-controlled manner, such as time-controlled and with suitable signaling via, for example, the display means and/or acoustic and/or haptic signal generators and/or by signaling means and/or alarms in the controller, for example, the smartphone app. This allows the administration device to be operated temporarily or permanently autonomously.

The administration device transmits the contents of the memories and/or the status of the administration device individually or as a whole to the controller periodically and/or spontaneously and/or after a discharge and/or upon request by the controller.

The invention further relates to a method, such as a computer-assisted method for the guided operation of an administration device for administering a medication, having a plurality of the steps:
Providing a default value for a discharge quantity and a predefined threshold duration on a controller
Transmitting the default value and the predefined threshold duration from the controller to the administration device or the electronic controller
Setting a pre-selected quantity on the administration device
Determining actuation states of an operating means which can release or operate a discharge of medication
Actuating the operating means on the administration device, where ejection of medication takes place as a discharge
Running measurement of a duration from the end of the discharge until the operating means is not actuated, or in other words, the time measurement runs from the end of the discharge as long as the actuating state of the operating means is actuated.

The method for the guided operation of an administration device for administering a medication is may be provided by additionally having one or more of the following steps:
Signaling the relation of pre-selected quantity to default value by the administration device, such as signaling that the pre-selected quantity is identical to the default value and/or that the administration device is ready for an administration
Signaling that a default value has been received or changed, for example, by a display and/or an acoustic signal generator
Establishing the duration from the end of the discharge until the operating means is not actuated as a holding duration
Creating a wireless authenticated pairing of the administration device and the controller, such as by an “out of band” method, for example, by means of a QR code on the administration device or the medication container insertable therein
Creating a cryptographically secured wireless connection between the administration device and the controller
Comparing the stored discharge quantities, characterizations, default values, statuses, parameters, real-time clocks, geographical coordinates between the administration device and the controller
Transmitting a specified dwell time from the controller to the administration device or the electronic controller
Signaling that the currently set pre-selected quantity is smaller than the default value
Signaling that the currently set pre-selected quantity is equal to the default value
Signaling that the currently set pre-selected quantity is greater than the default value
Signaling the end of the discharge as soon as the set pre-selected quantity or a discharge quantity is discharged
Signaling that the duration does not reach the predefined threshold duration
Signaling that the duration reaches or exceeds the predefined threshold duration
Signaling that the duration does not reach a specified dwell time
Signaling that the duration reaches or exceeds a specified dwell time
Providing the discharge quantity with one or more of time stamp, medication designation, concentration, lot number, carpule number, temperature, geographical location, spatial orientation, etc.
Characterizing or identifying the discharge quantity as a priming quantity if the duration is less than the predefined threshold duration
Characterizing or identifying the discharge quantity as an administration quantity if the duration is greater than or equal to the predefined threshold duration
Characterizing or identifying the discharge quantity as a compliant administration quantity if the duration is greater than or equal to the specified dwell time
Deleting the default value if the administration quantity is equal to or greater than the default value
Storing the administration quantity in the administration device
Storing the priming quantity in the administration device
Transmitting the administration quantity from the administration device to the controller
Transmitting the priming quantity from the administration device to the controller
Retrieving a confirmation on the controller for verifying the last administration quantity and/or priming quantity

For use cases in which the administration quantity is smaller than the default value, the method for the guided operation of an administration device for administering a medication is may be provided by further having one or more of the following steps:
Decreasing the default value by the administration quantity
Transmitting the decreased default value to the controller
Request on the controller to change the container, e.g., a carpule in the administration device
Request on the controller to perform priming on the administration device

Such a use case can arise if the residual quantity of medication in the container is not sufficient to make a pre-selected quantity or discharge quantity in the amount of the default value. In this case, the container or the carpule must be changed. In another use case, the user or the patient can reach a default value by means of two or more administrations—intentionally or because a first administration is terminated early by a device error.

The invention further relates to the method, such as the computer-assisted method for the guided operation of an administration device for administering a medication carried out on an administration device for an administration device for a medication and/or carried out on an administration device for a medication.

Further aspects of preferred developments and their embodiments are documented in European application EP 21187086.0, which is incorporated by reference herein in its entirety for any useful purpose.

### BRIEF DESCRIPTION OF THE DRAWINGS

FIG. 1 schematically shows an embodiment of the administration device according to the invention for a medication in a device or system environment;

FIGS. 2a and 2b show two isometric views of an embodiment of the administration device according to the invention in an automatic pen;

FIGS. 3a-3c show various views of the administration device in an automatic pen;

FIG. 4 shows nine operating states of an embodiment of the administration device;

FIG. 5 shows components of an exemplary system environment of an embodiment of the administration device according to the invention; and

FIG. 6 shows by way of example a use case of a guided operation with an administration device according to the invention in an exemplary system environment with associated use actions and data flows.

### DETAILED DESCRIPTION

### Definitions

The term “administration device” includes injection devices which can discharge certain doses of medication. Multi-use injection devices usually contain a container for the medication and a dose setting device which allows the user to pre-select a desired dose for each individual discharge. Furthermore, multi-use injection devices are equipped or can be equipped with a needle having a hollow cannula, through which the medication can be discharged. Insulin pens are examples of such multi-use injection devices which are known to be used for the treatment of diabetes mellitus. Insulin pens contain a carpule with an insulin-containing solution which is sufficient for a plurality of discharges in such a way that a carpule filling can be sufficient for several days. Such insulin pens allow the user to pre-select and correspondingly discharge a dose specific for each discharge. Automatic pens have a motor drive.

The term “product”, “medication”, or “medical substance” in the present context includes any flowable medical formulation which is suitable for controlled administration by means of a cannula or hollow needle in subcutaneous or intramuscular tissue, for example a liquid, a solution, a gel, or a fine suspension containing one or more medical active ingredients. A medication can thus be a composition with a single active ingredient or a premixed or co-formulated composition with a plurality of active ingredients from a single container. The term includes drugs, such as peptides (e.g., insulins, insulin-containing medications, GLP 1-containing preparations as well as derived or analogous preparations), proteins and hormones, biologically obtained or active ingredients, active ingredients based on hormones or genes, nutrient formulations, enzymes, and other substances both in solid (suspended) or liquid form. The term also includes polysaccharides, vaccines, DNA or RNA or oligonucleotides, antibodies or parts of antibodies as well as suitable base substances, excipients, and carrier substances.

The term “distal” refers to a side or direction directed toward the front, piercing-side end of the administration apparatus or toward the tip of the injection needle. In contrast, the term “proximal” refers to a side or direction directed toward the rear end of the administration apparatus that is opposite the piercing-side end.

In the present disclosure, the terms “injection system” or “injector” or “pen” are understood to mean a device in which the injection needle is removed from the tissue after a controlled quantity of the medical substance has been dispensed, optionally after a dwell time has elapsed during which the medical substance can diffuse into the tissue. A specific dwell time can, for example, be specified on the patient information leaflet for the medication. In contrast to an infusion system, the injection needle in an injection system or in an injector thus does not remain in the tissue for a longer period of several hours.

Aspects of the present disclosure are described below in connection with the accompanying figures. These embodiments are intended to show basic possibilities and the effectiveness of the invention by way of example and are in no way to be interpreted as limiting.

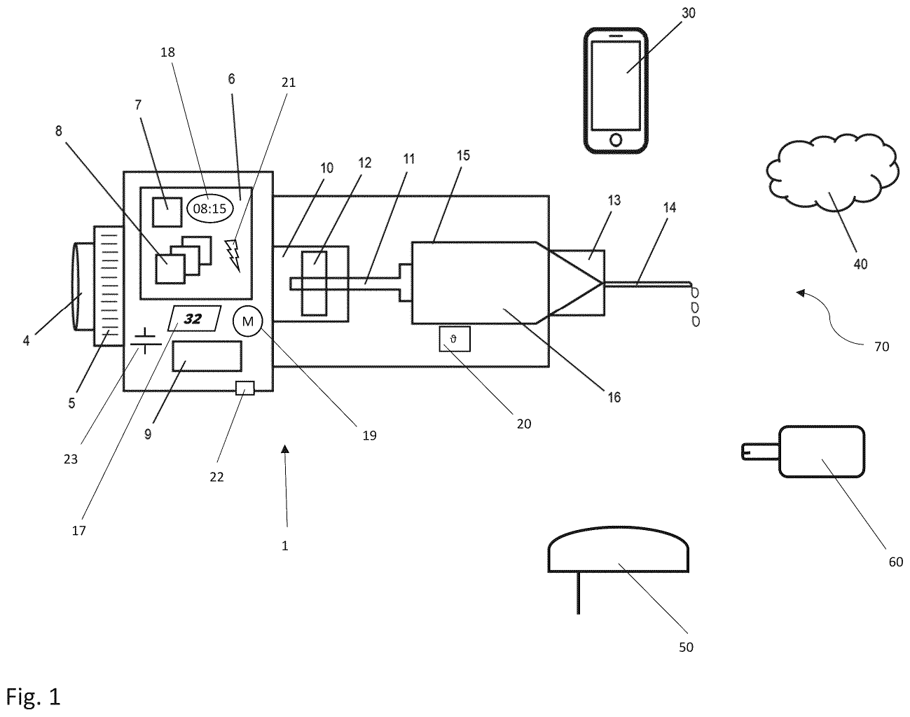

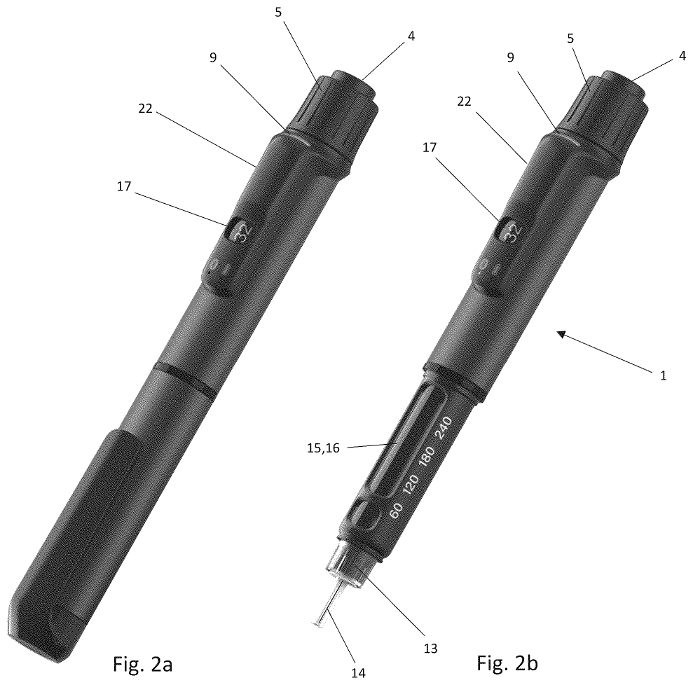

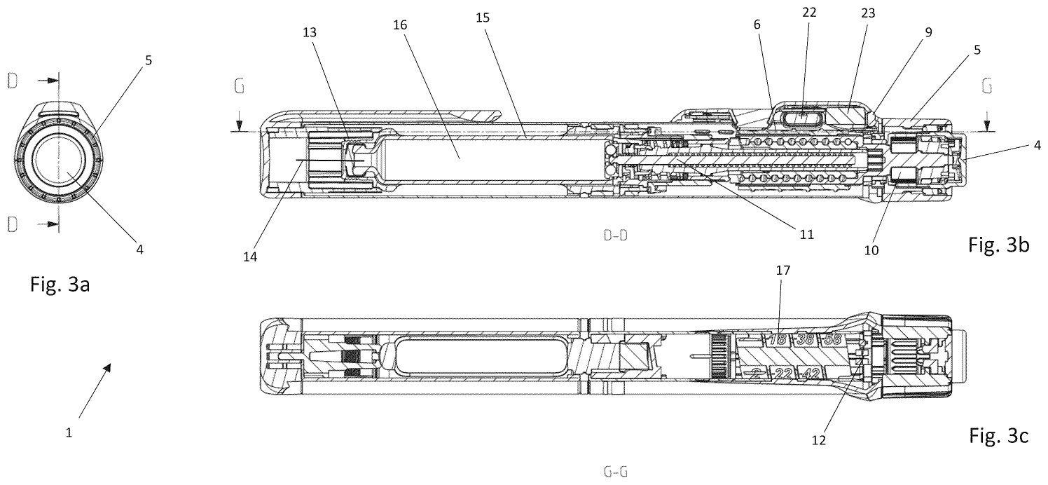

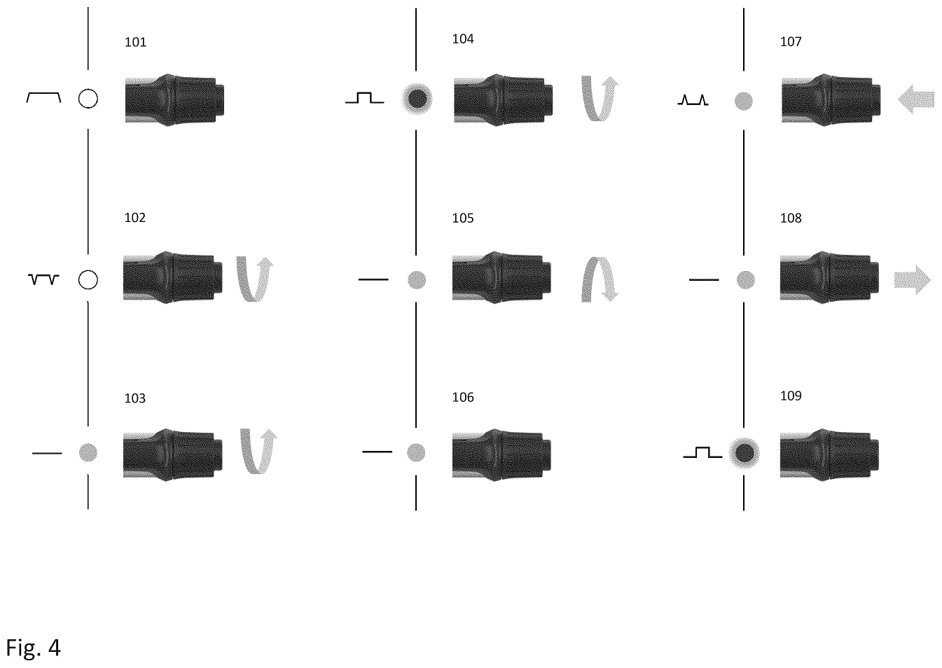

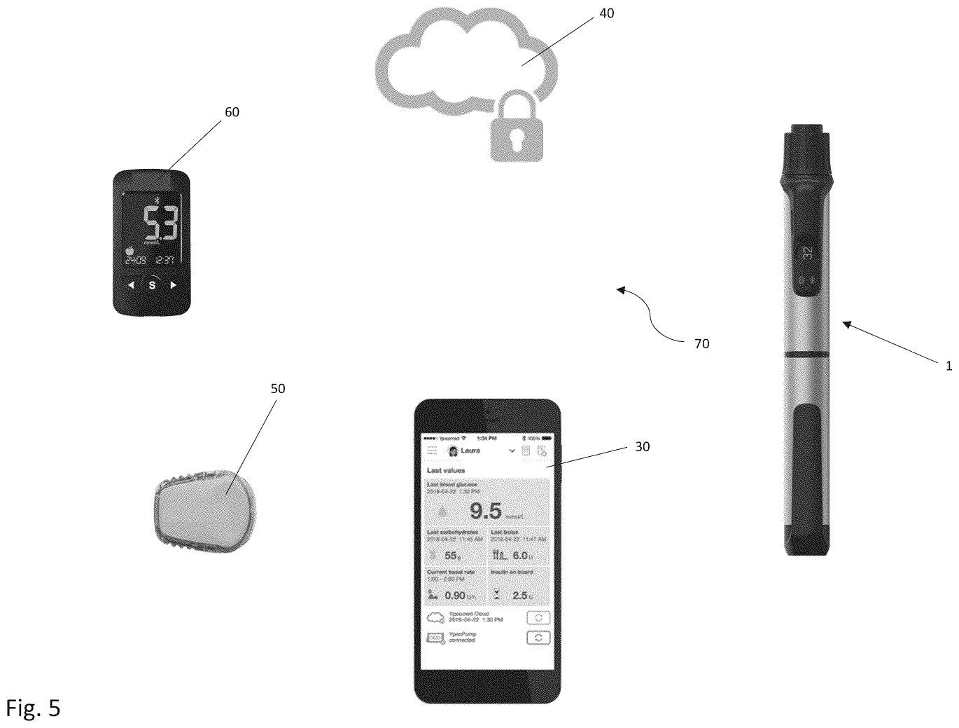

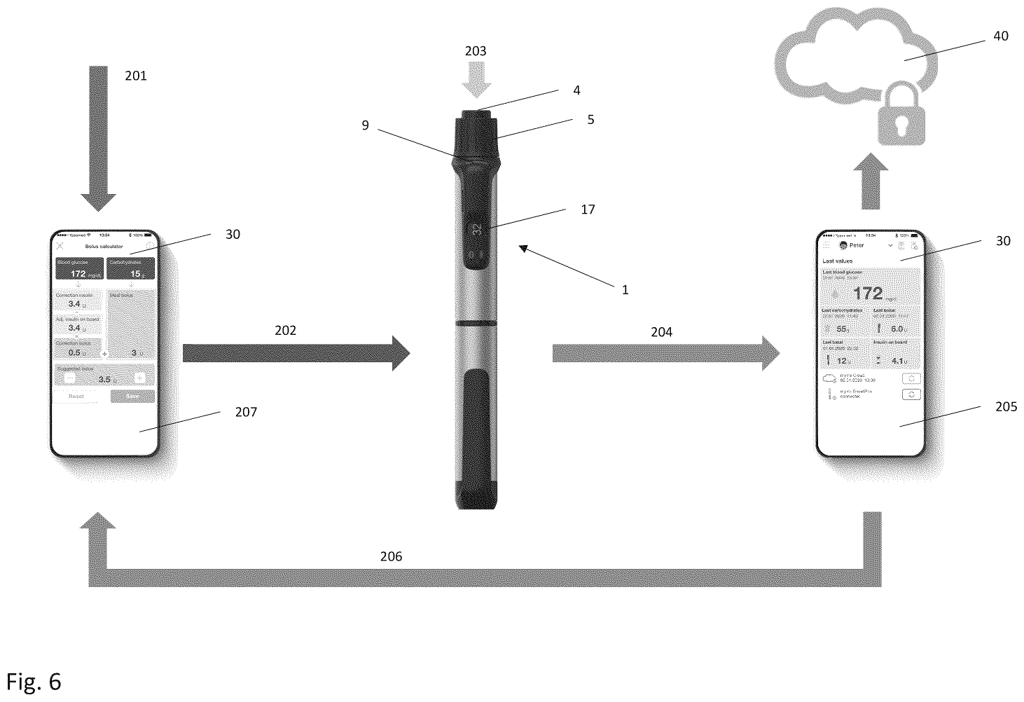

Turning to the Figures, FIG. 1 schematically shows an embodiment of the administration device 1 according to the invention. A pre-selection means 5, for example, in the form of a rotary ring, serves to set or correct a dose to be discharged. The currently set dose can be read on a scale 17, for example, by means of printed digits on an adjusting sleeve through a viewing window in the administration device 1. An operating means 4, for example, in the form of a pushbutton 4 that can be actuated in the axial direction at the proximal end of the administration device 1, serves to discharge the set dose. For this purpose, a drive 10, for example, a motor or a screw gear, is switched on or released or actuated by the operating means 4. The drive 10 can thereby act on an output means 11, for example, a piston rod with a distally attached flange. As a result, for a discharge of medication 16, the output means 11 can move axially in the distal direction relative to a container 15 inserted in the administration device 1, for example, a carpule with a stopper and septum, where the medication 16 is pressed through a hollow cannula 14 in the needle 13, which is inserted into the septum, by means of the stopper movement until the drive 10 is stopped, for example, in that a zero stop is reached or the set dose is discharged or the pressable operating means 4 is released. The administration device 1 has an electronic controller 6, for example, an electronic assembly containing a microprocessor, a memory 8 for programs and data, a timer or time measuring device 7 with a real-time clock 18 and a transceiver 21 for wireless communication, for example, by means of BLE technology. The timer or time measuring device 7 is configured such that a duration from the end of a discharge to the release of the operating means 4 can be continuously measured, where the current running value of the duration can be read out at any time in an event- or program-controlled manner, and the reached value of the duration is available. The relation of the duration to a specified threshold can currently be determined at any time in order to determine whether a discharge that has taken place is to be characterized as a priming or as an administration and/or the measured duration can be used currently or retrospectively to determine whether an administration is compliant with the intended use in relation to the dwell time. The electronic controller 6 can qualitatively and/or quantitatively detect the rotational and/or axial movements of the drive 10, the pre-selection means 5 and the operating means 4 by means of one or more sensors 12. Alternatively, the actuation of the operating means 4 can also be determined by means of touch sensors on a, for example, capacitive, resistive or piezoelectric basis or by means of an electromechanical switch. The administration device 1 further includes a display means 9, for example, in the form of a multicolored (RGB) LED and an associated light guide, the outer surface of which is embedded in the outer side of the housing of the administration device 1 in a manner that is well visible. In addition, the electronic controller 6 can activate a vibration alarm 19 and measure and register the temperature by means of one or more connected temperature sensors, for example, a temperature sensor 20 in the administration device 1 can be thermally connected to the container 15 or to the medication 16. The electronic controller 6 is fed by an energy source 23 installed in the administration device, which can be formed, for example, from one or more lithium ion elements. A connection 22, for example, a USB-C socket, is used to charge the energy source and/or to download or upload programs and/or configuration data and/or use data. A device or system environment is also shown by way of example in FIG. 1 and FIG. 5. This can have a remote control or smartphone 30 which contains one or more apps, e.g. for keeping a therapeutic log and for calculating dose recommendations. Furthermore, the system environment can have one or more of host/PC/cloud computer 40, continuous glucose monitor 50, and glucose monitor 60. The devices of the system environment may be communicatively coupled to one another, such as to the administration device 1, wirelessly. One or more of the devices 30, 40, 50, 60 can function as a controller 70, e.g., as a system controller, for the administration device 1. In the present example, the smartphone 30 also assumes the controller function as a controller 70. FIGS. 2a and 2b show two isometric views of an embodiment of the administration device 1 according to the invention in a spring-driven automatic pen with a protective cover (FIG. 2a) and without a protective cover (FIG. 2b). The desired dose can be set by means of the rotary ring 5, where a drive spring is simultaneously tensioned. The currently set or pre-selected dose can be read on the scale 17. By actuating the pushbutton 4, the administration device 1 is activated, where medication 16 is discharged from the container 15 through the cannula 14 in the needle 13. The display 9 conveys to the user/the patient information about the currently set dose, the status of the running discharge, and information regarding the operating state of the administration device 1 or the system environment. FIGS. 3a, 3b and 3c show the administration device 1 or the automatic pen in a proximal view (FIG. 3a) and in two longitudinal sections D-D (FIG. 3b) and G-G (FIG. 3c). In addition to the elements, already described in FIGS. 2a and 2b, push button 4, rotary ring 5, carpule 15 with medication 16, needle 13 with cannula 14, FIGS. 3a-3c show the electronic controller 6 with the display 9, the USB socket 22 and the battery 23, the spring motor 10, the piston rod 11, the printed scale sleeve 17 and a sensor group 12 connected to the electronic controller 6 for rotational and axial movements of parts of the administration device 1. FIG. 4 shows nine operating states 101 to 109 on the basis of an exemplary embodiment of the administration device 1 in an automatic pen. State 101 shows the administration device 1 after successfully switching on. Such a switch-on process can be effected by manipulating, for example, the rotary ring 5 or the pushbutton 4. As a result, the electronic controller 6 is switched on and/or awaken from an energy-saving mode, which is signaled via the display 9, for example, in that it is illuminated continuously (uninterrupted) white. As soon as the administration device 1 is switched on and connected to the controller 70, the device can receive a dose recommendation or a default value from the controller. The state 102 shows the administration device 1 when a dose is filled (increased) by rotating the rotary ring 5 clockwise. The display 9 signals the latching steps during the filling, for example, by briefly interrupting the white illumination per dose step or dose unit. The state 103 shows the administration device 1 when the dose recommendation or the default value is reached by the set dose. The display 9 indicates that the default value is reached and signals to the user or the patient that the recommended dose is reached, for example, by the display 9 being illuminated continuously green in this case. With a further increase by one latching step, the display changes to white in order then, in the event of a further increase or a clear exceeding of the default value as shown in state 104, to change to red after, for example, a further two latching steps or dose units. In state 105, this dose that is pre-selected to be too high is corrected by rotating the rotary ring 5 counterclockwise, which is guided or acknowledged by the signaling by means of the display 9 in that the display changes from red to white to green again. This is shown in state 106, where the dose set on the administration device 1 matches the default value, and the display is illuminated continuously green, for example. The automatic pen can be inserted at the injection site. The discharge can then be initiated as shown for state 107 by pressing the pushbutton 4. The running discharge is signaled, for example, by the display flashing green. After the end of the discharge, this signaling is continued during a specified dwell time for, for example, a further 10 seconds. As shown for state 108, a discharge can only be evaluated as a compliant administration after this dwell time has elapsed, and this is signaled, for example, by green continuous light, so that the pushbutton 4 can be released and the pen can be removed from the injection site. If errors occur during use of the administration device 1, such errors are signaled as shown for operating state 109, for example, by red illumination or flashing of the display 9. FIG. 6 shows, by way of example, a use case of a guided operation with an administration device 1 according to the invention in an exemplary system environment with associated use actions and data flows. The guided operation is described on the basis of an administration of insulin in the context of diabetes treatment and on the basis of FIG. 4. In addition to the administration device 2 with the administration device 1, the system environment has the following components: a smartphone 30 with apps which implement a diabetes log 205 and a bolus calculator 207, and a cloud 40 and/or a host computer which, for example, can deliver, process or secure treatment-related or patient-specific data. The use case can thus begin in that treatment-related input data 201 are retrieved by the bolus calculator 207 by means of a user interface on the smartphone 30, for example, as a current glucose value and as the energy content of an immediately imminent meal. The bolus calculator 207 calculates a bolus recommendation 202 from this and from further parameters, for example, the time of day, or from the discharges already recently performed. This can be confirmed by a dialog by means of a user interface on the smartphone 30 and is then transmitted as a default value to the administration device 1. A patient can thereupon fill the desired dose, guided on the rotary ring 5, via the display 9 and the scale 17 and discharge it by actuating the pushbutton 4. If, after a completed discharge, the duration or dwell time of the needle in the tissue is equal to or greater than a predefined threshold duration, the discharge can be transmitted to the diabetes log 205 as a treatment-related administration quantity 204 with a date and time stamp. From this and the preceding entries in the diabetes log, an “insulin on board” IOB value 206 can then be calculated continuously or at the point in time of a next bolus calculation.

### LIST OF REFERENCE SIGNS

1 Administration device
4 Operating means, pushbutton
5 Pre-selection means, rotary ring
6 Electronic controller or control unit
7 Timer or time measuring device
8 Memory
9 Display means
10 Drive, spring motor
11 Output means, piston rod
12 Sensor
13 Needle
14 Hollow cannula
15 Container, carpule
16 Medication
17 Scale for pre-selection means
18 Real-time clock
19 Vibration alarm
20 Temperature sensor
21 Transceiver
22 Connection, charging socket
23 Energy source, battery
30 Smartphone, app(s)
40 Cloud/host/PC
50 CGM device
60 BGM device
70 Controller (or system controller)
101 Device switched on
102 Select dose
103 Default value confirmed
104 Default value exceeded
105 Default value confirmed
106 Insert needle
107 Discharge and/or dwell time running
108 Administration completed
109 Error signal
201 Treatment-related input data
202 Bolus recommendation or default value
203 Injection is performed
204 Discharge quantity with time stamp and characterization (identification)
205 Log/last administration quantity
206 Insulin on board (IOB)
207 Bolus calculator

What is claimed is:

1. An administration device for parenteral administration of a medication, comprising:
a pre-selection means by means of which a pre-selected quantity can be mechanically or electronically set and/or stored;
a manually operable drive or a releasable motor drive configured to move an output means relative to a container arranged in the administration device and containing a medication to cause a discharge quantity to be discharged from the container through a needle and/or a hollow cannula, the discharge quantity corresponding to the pre-selected quantity;
an operating means configured to be manually actuated having a non-actuated state and an actuated state, the operating means configured to operate and/or release the drive in the actuated state; and
a control unit comprising a timer, wherein the timer is configured such that, when the operating means is actuated, the timer continuously measures a duration of time between when the output means is no longer moved by the drive and when the operating means is transferred to the non-actuated state, and wherein the control unit is configured to identify the discharge quantity as a priming quantity if the duration is shorter than a predefined duration threshold.

2. The administration device of claim 1, wherein the control unit is configured to identify the discharge quantity as an administration quantity if the duration is equal to or greater than the predefined duration threshold.

3. The administration device of claim 2, wherein the predefined duration threshold ranges from 1 to 20 seconds, from 2 to 5 seconds, is 3 seconds, or is variably specified from a system controller.

4. The administration device of claim 3, wherein the system controller is selected from the group consisting of: a remote control, a smartphone application, a blood glucose monitor, a continuous glucose monitor, a personal computer, a network, a cloud system, a host system, is implemented in the administration device, or a combination of two or more thereof.

5. The administration device of claim 3, wherein the control unit further comprises a real-time clock configured to be synchronized with a clock of the system controller.

6. The administration device of claim 3, wherein the control unit further comprises a transceiver configured to establish a wireless connection to one or more of: the control system, a remote control, a smartphone application, a blood glucose meter, a continuous glucose monitor, a personal computer, a network, a cloud system, a host system, or a combination of two or more thereof.

7. The administration device of claim 6, wherein the control unit is configured to establish the wireless connection with the control system and to receive a default value from the control system for a next discharge over the wireless connection.

8. The administration device of claim 7, wherein the administration device comprises a display means communicatively coupled to the control unit, wherein the control unit is configured to cause the display means to signal that a current pre-selected quantity set with the pre-selection means is at least one of: smaller than, equal to, somewhat greater than, or much greater than the default value.

9. The administration device of claim 8, wherein the administration device comprises a scale configured to display the current pre-selected quantity from the pre-selection means, or wherein the current pre-selected quantity from the pre-selection means is caused to be displayed by the control system, or both.

10. The administration device of claim 2, wherein the control unit further comprises a memory configured to store at least the discharge quantity as an administration quantity upon the discharge quantity not being identified as the priming quantity, and wherein at least one of the discharge quantity, the administration quantity, or the priming quantity is provided with at least one of: a time stamp, a date stamp, or at least one further characterization.

11. The administration device of claim 1, wherein the control unit is configured to determine a movement or an absence of movement of the output means based on one or more drive parameters or based on information received from one or more of: a limit switch, a sensor, a rotary encoder, or a linear encoder.

12. The administration device of claim 1, wherein the control unit further comprises a sensor, and wherein the controller is configured to determine a quantity of medication based on a sensed movement of the output means.

13. A method of controlling an administration device for administering a medication, comprising the steps of:
providing a default value for a discharge quantity and a predefined duration threshold on a control system;
transmitting, by the control system, the default value and the predefined duration threshold to the administration device;
setting a pre-selected quantity on the administration device;
determining actuation states of an operating means, the operating means configured to release or operate a discharge of medication;
measuring a duration of time between an end of the discharge and when the operating means is not actuated;
identifying the discharge quantity as a priming quantity if the duration is less than the predefined duration threshold.

14. The method of claim 13, further comprising one or more of the following steps:
signaling a relation of the pre-selected quantity to the default value by the administration device, comprising signaling that the pre-selected quantity is identical to the default value, or signaling that the administration device is ready for an administration, or both;
generating a wireless authenticated pairing of the administration device and the control system, comprising pairing by an “out of band” method using a QR code on the administration device or a medication container insertable therein;
generating a cryptographically secured wireless connection between the administration device and the control system;
comparing stored discharge quantities, characterizations, default values, statuses, parameters, real-time clocks, geographical coordinates between the administration device and the control system;
signaling that a currently set pre-selected quantity is smaller than the default value;
signaling that the currently set pre-selected quantity is equal to the default value;
signaling that the currently set pre-selected quantity is greater than the default value;
signaling the end of the discharge upon the currently set pre-selected quantity or the discharge quantity is discharged;
signaling that the duration does not reach the predefined duration threshold;
signaling that the duration reaches or exceeds the predefined duration threshold;
signaling that the duration does not reach a specified dwell time;
signaling that the duration reaches or exceeds the specified dwell time;
determining the duration from the end of the discharge until the operating means is not actuated as a holding duration;
providing the discharge quantity with one or more of a time stamp, a medication designation, a concentration, a lot number, a carpule number, a temperature, a geographical location, or a spatial orientation;
identifying the discharge quantity as an administration quantity if the duration is greater than or equal to the predefined threshold duration;
identifying the discharge quantity as a compliant administration quantity if the duration is greater than or equal to the specified dwell time;
deleting the default value in the administration device if the administration quantity is equal to or greater than the default value;
storing the administration quantity in the administration device;
storing the priming quantity in the administration device;
transmitting the administration quantity from the administration device to the system controller;
transmitting the priming quantity from the administration device to the system controller; or
retrieving a confirmation on the system controller for verifying a last administration quantity and/or the priming quantity.

15. The method of claim 14, further comprising one or more of the following steps:
decreasing the default value by the administration quantity in the administration device;
transmitting the decreased default value from the administration device to the system controller;
requesting by the system controller to change the container in the administration device; or
requesting by the system controller to perform a priming on the administration device.

16. The method of claim 13, wherein the administration device comprises:
a pre-selection means by means of which the pre-selected quantity can be mechanically or electronically set and/or stored;
a manually operable drive or a releasable motor drive configured to move an output means relative to a container arranged in the administration device and containing the medication to cause the discharge quantity to be discharged from the container through a needle and/or a hollow cannula, the discharge quantity corresponding to the pre-selected quantity; and
a control unit configured to identify the discharge quantity as the priming quantity,
wherein the operating means is configured to be manually actuated, the operating means having a non-actuated state and an actuated state and configured to operate and/or release the drive in the actuated state.
